(19) United States
(12) Patent Application Publication
(10) Pub. No.: US 2024/0122456 A1
(43) Pub. Date: Apr. 18, 2024

(54) AN OROPHARYNGEAL GLOVE FOR USE WITH RIGID AND FLEXIBLE BRONCHOSCOPES, AND METHODS

(71) Applicant: Ismael A. Matus, Landenberg, PA (US)

(72) Inventor: Ismael A. Matus, Landenberg, PA (US)

(73) Assignee: Skillhead LLC, Buford, GA (US)

(21) Appl. No.: 18/009,664

(22) PCT Filed: Oct. 18, 2022
(86) PCT No.: PCT/US2022/078304

§ 371 Date: Dec. 9, 2022

(51) Int. Cl. A61B 1/00; A61B 1/267; A61M 16/04; A61M 16/08

(52) U.S. Cl. CPC A61B 1/00135; A61B 1/2676; A61M 16/0459; A61M 16/0493; A61M 16/0816; A61M 16/0883; A61M 2202/0208; A61M 2210/065

(57) ABSTRACT

An oropharyngeal glove (OPG) is provided for use in a rigid, or flexible bronchoscopy procedure and for anesthesia recovery. Portions of the OPG conform to portions of the patients mouth and throat. During a bronchoscopy procedure, the bronchoscope tube passes through an opening in a proximal end of the OPG, through the OPG and through an opening in a distal end of the OPG into the patient s trachea. A flexible body of the OPG protects the patients mouth, throat and vocal cords from being damaged by the bronchoscope tube. A tubular extension disposed on the proximal end of the OPG provides the opening through which the bronchoscope tube first passes. Post procedure, the tubular extension can act as an airway device that connects to a ventilator machine to deliver air to the patient, thereby obviating the need to install a separate airway device to ventilate the patient during anesthesia recovery.

### CROSS-REFERENCE TO RELATED APPLICATIONS

The present application is a continuation-in-part (CIP) application of U.S. application Ser. No. 17/511,155, filed on Oct. 26, 2021, entitled “AN OROPHRANGEAL GLOVE FOR USE WITH RIGID AND FLEXIBLE BRONCHSCOPES, AND METHODS,” which is currently pending, and which is incorporated by reference herein in its entirety.

### TECHNICAL FIELD

The present disclosure relates to bronchoscopy, which is an endoscopic process in which an instrument known as a bronchoscope is inserted through the patient's mouth into the airways to allow the physician to examine the airways for abnormalities such as bleeding, tumors or inflammation, for example. More particularly, the present disclosure relates to an oropharyngeal glove for use with rigid and flexible bronchoscopes and methods for using the glove.

### BACKGROUND

Bronchoscopy is an endoscopic process that involves visualizing the inside of the airways for therapeutic and diagnostic purposes. An instrument known as a bronchoscope is inserted through the patient's mouth into the airways to allow the physician to examine the airways for abnormalities such as bleeding, tumors or inflammation, for example.

Generally, there are two types of bronchoscopes: flexible bronchoscopes and rigid bronchoscopes. Flexible bronchoscopes have a fiber optic system that transmits an image from the end of an optical fiber that is inside of the patient to an eyepiece or camera at the opposite end of the optical fiber. Rigid bronchoscopes have a larger lumen than that of the flexible bronchoscope and are typically made of a hard metallic material.

There are problems that can occur when performing rigid bronchoscopy. Because of the rigid nature of the instrument, it can sometimes cause abrasions or lacerations to the patient's mouth, throat or vocal cords and can damage the patient's teeth.

Also, as rigid bronchoscopic procedures are performed under general anesthesia and are considered “open circuit” procedures, varying degrees of air leaks exist not only through the barrel of the rigid bronchoscope to the atmosphere, but also from the patient's airway around the rigid bronchoscope which can challenge ventilation of the patient.

In addition to the problems discussed above, both rigid and flexible bronchoscopy procedures require use of a separate airway device post procedure for ventilating the patient while the patient recovers from anesthesia. During a rigid bronchoscopy procedure, air is supplied to the patient's airway through the tube of the rigid bronchoscope. Once the procedure is completed, the rigid bronchoscope is removed and an airway device, such as an Igel® supraglottic airway device with mask, an endotracheal tube or other type of airway device, that is connected to a ventilator is attached to the patient to resume ventilation of the patient. During a flexible bronchoscopy procedure, such a mask or endotracheal tube is used and the tube of the flexible bronchoscope is fed into the patient through an uncapped port of the airway device. When the bronchoscopy procedure is complete, the flexible bronchoscope tube is removed from the patient and from the airway device and the uncapped port of the airway device is capped. Air delivered by the airway device continues to ventilate the patient until the patient is recovered.

There is significant cost associated with purchasing the separate airway device and having a healthcare professional apply it to the patient. A need exists for a medical device that can be used when performing bronchoscopy to protect the patient's mouth, throat, vocal cords and teeth, that reduces or eliminates the possibility of air leakage during the procedure and that obviates the need for a separate airway device to perform post-procedure anesthesia recovery.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a front perspective view of the oropharyngeal glove in accordance with a representative embodiment.

FIG. 2A is a left side view of the oropharyngeal glove shown in FIG. 1.

FIG. 2B is a side cross-sectional view of the oropharyngeal glove shown in FIG. 2A taken along section lines B-B′ shown in FIG. 2A.

FIG. 2C is an expanded view of the distal portion of the oropharyngeal glove shown in FIG. 2B in the circle labeled “C” in FIG. 2B.

FIG. 3A is a proximal end view of the oropharyngeal glove shown in FIG. 1.

FIG. 3B is a side cross-sectional view of the oropharyngeal glove shown in FIG. 3A taken along section lines A-A′ shown in FIG. 3A.

FIG. 4 is a photograph of a prototype of the oropharyngeal glove shown in FIGS. 1-3B in an insufflated state to form an airtight or nearly airtight seal between outer surfaces of the glove and a patient's mouth and throat.

### DETAILED DESCRIPTION

The present disclosure is directed to various representative embodiments of an oropharyngeal glove (OPG) for use with rigid and flexible bronchoscopes, as well as representative embodiment of methods of using the glove to perform rigid and flexible bronchoscopy procedures. Portions of the OPG conform to portions of the patient's mouth and throat. During a bronchoscopy procedure, the bronchoscope tube passes through an opening in a proximal end of the OPG, through the OPG and through an opening in a distal end of the OPG into the patient's trachea. A protective lining of the OPG protects the patient's mouth, throat and vocal cords from being damaged by the bronchoscope tube. A tubular extension disposed on the proximal end of the OPG provides the opening through which the bronchoscope tube first passes into the OPG. Post procedure, the tubular extension can act as an airway device that connects to a ventilator machine via a breathing circuit to deliver air to the patient, thereby obviating the need to install a separate airway device to ventilate the patient during anesthesia recovery.

In the following detailed description, for purposes of explanation and not limitation, exemplary, or representative, embodiments disclosing specific details are set forth in order to provide a thorough understanding of the inventive principles and concepts. However, it will be apparent to one of ordinary skill in the art having the benefit of the present disclosure that other embodiments according to the present teachings that are not explicitly described or shown herein are within the scope of the appended claims. Moreover, descriptions of well-known apparatuses and methods may be omitted so as not to obscure the description of the exemplary embodiments. Such methods and apparatuses are clearly within the scope of the present teachings, as will be understood by those of skill in the art. It should also be understood that the word “example,” as used herein, is intended to be non-exclusionary and non-limiting in nature.

The terminology used herein is for purposes of describing particular embodiments only and is not intended to be limiting. Any specifically-defined terms are in addition to the technical, scientific, or ordinary meanings of the defined terms as commonly understood and accepted in the relevant context.

The terms “a,” “an” and “the” include both singular and plural referents, unless the context clearly dictates otherwise. Thus, for example, “a device” includes one device and plural devices. The terms “substantial” or “substantially” mean to within acceptable limits or degrees acceptable to those of skill in the art. For example, the term “substantially parallel to” means that a structure or device may not be made perfectly parallel to some other structure or device due to tolerances or imperfections in the process by which the structures or devices are made. The term “approximately” means to within an acceptable limit or amount to one of ordinary skill in the art.

Relative terms, such as “over,” “above,” “below,” “top,” “bottom,” “front,” “back,” “upper” and “lower” may be used to describe the various elements' relationships to one another, as illustrated in the accompanying drawings. These relative terms are intended to encompass different orientations of the device and/or elements in addition to the orientation depicted in the drawings. For example, if the device were inverted with respect to the view in the drawings, an element described as “above” another element, for example, would now be below that element.

The term “bronchoscope,” as that term is used herein, can mean a rigid bronchoscope or a flexible bronchoscope unless specifically referred to herein as a “rigid bronchoscope” or a “flexible bronchoscope.” A “rigid bronchoscope,” as that term is used herein, means a bronchoscope having a rigid tube, as is known in the art of bronchoscopy. A “flexible bronchoscope,” as that term is used herein, means a bronchoscope having a flexible tube, as is known in the art of bronchoscopy.

When the oropharyngeal glove (referred to herein as “the OPG”) is in its installed state, it can be insufflated to conform to the patient's mouth and throat to provide an airtight, or nearly airtight, seal between the OPG and the patient's mouth and through to prevent or greatly reduce air leakage. In preferred embodiments, the OPG includes upper and lower teeth guards that are in contact with the front sides of the patient's upper and lower front teeth, respectively, when the OPG is in the installed state. The OPG has a first opening disposed in its proximal end that allows the bronchoscope tube to enter the OPG. The OPG has a second opening formed in its distal end through which the bronchoscope tube passes to enter the patient's trachea. The portions of the OPG that conform to the patient's mouth and throat comprise a protective lining that protects the mouth, throat and vocal cords from being damaged by the rigid bronchoscope. The upper and lower teeth guards protect the patient's upper and lower front teeth, respectively, from being damaged by the bronchoscope tube during the bronchoscopy procedure and during installation and removal of the bronchoscope. It should be noted that the teeth guards are preferred, but not required, in cases where the OPG is being used with a rigid bronchoscope having a rigid tube to protect the teeth, but the teeth guards may not be needed and therefore may not be part of the OPG when the OPG is being used with a flexible bronchoscope having a flexible tube.

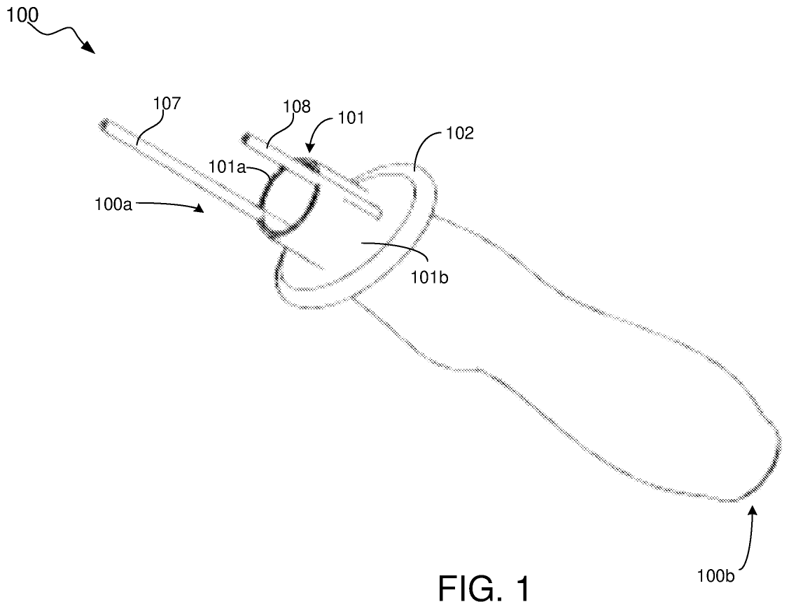

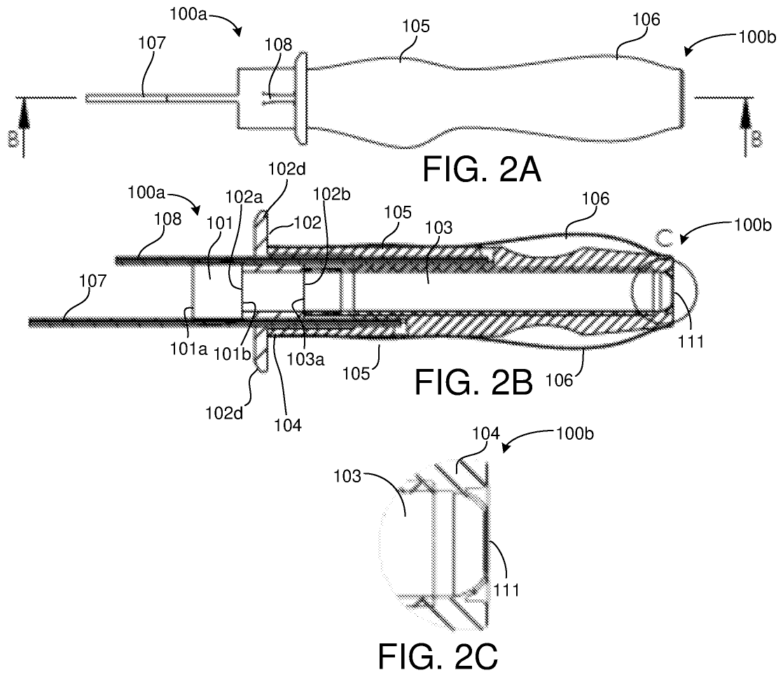

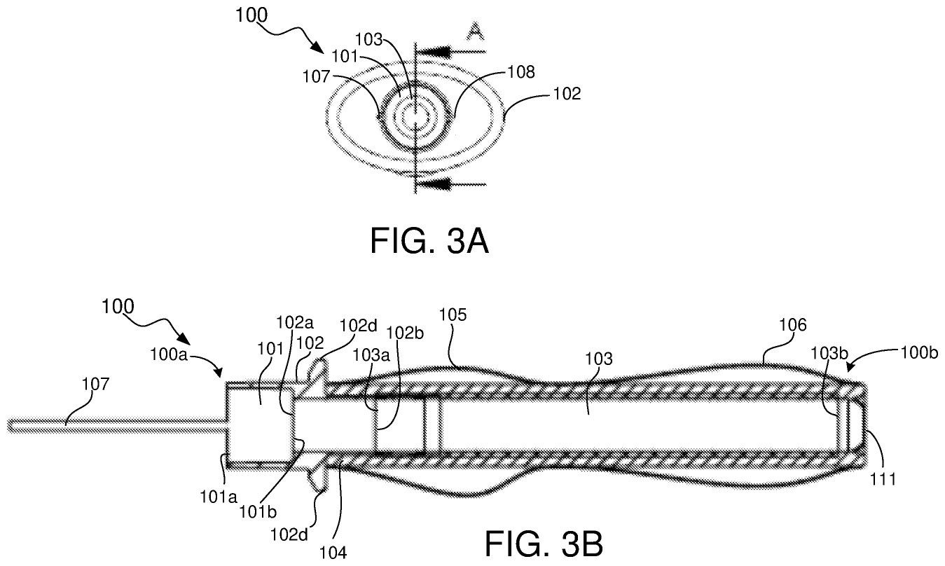

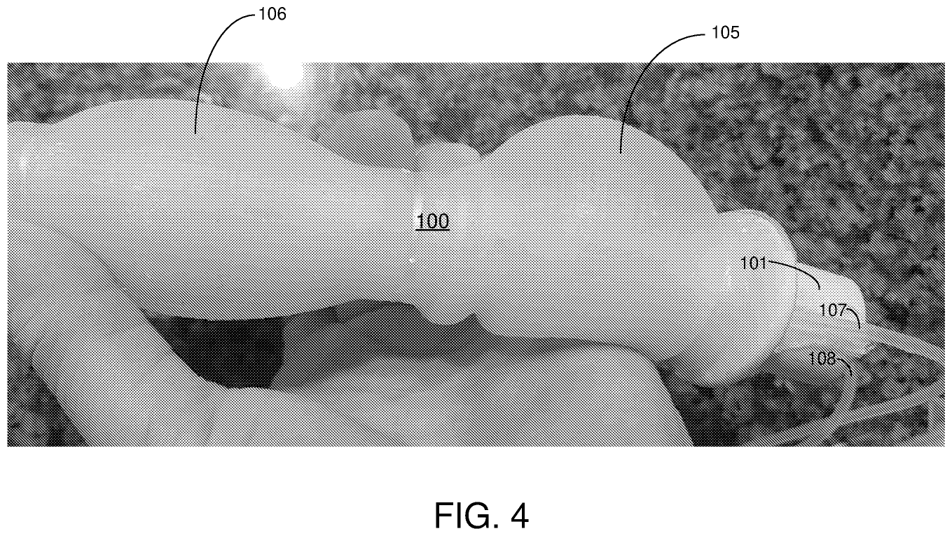

FIG. 1 is a front perspective view of the OPG 100 in accordance with a representative embodiment. FIG. 2A is a left side view of the OPG 100 shown in FIG. 1. FIG. 2B is a side cross-sectional view of the OPG 100 shown in FIG. 2A taken along section lines B-B′ shown in FIG. 2A. FIG. 2C is an expanded view of the portion of the distal end 100b of the OPG 100 labeled “C” in FIG. 2B. FIG. 3A is an end view of the proximal end 100a of the OPG 100 shown in FIG. 1. FIG. 3B is a side cross-sectional view of the OPG 100 shown in FIG. 3A taken along section lines A-A′ of FIG. 3A. FIG. 4 is a photograph of the OPG 100 shown in FIGS. 1-3B in an insufflated state in which it would be place to form an airtight or nearly airtight seal between outer surfaces of the OPG 100 and inner surfaces of a patient's mouth and throat.

The proximal end 100a of the OPG 100 includes a tubular extension 101 that is adapted to be coupled with a breathing tube of a breathing circuit of a ventilator to enable the OPG 100 to be used after a bronchoscopy procedure as part of the airway circuit to continue ventilating the patient until the patient has recovered from anesthesia. By performing this additional anesthesia recovery function, the OPG 100 obviates the need for a separate airway device for performing anesthesia recovery. As indicated above, currently both rigid and flexible bronchoscopy procedures require use of a separate airway device post procedure for ventilating the patient while the patient recovers from anesthesia. Currently, during a rigid bronchoscopy procedure, air is supplied to the patient's airway through the rigid tube of the rigid bronchoscope. Once the bronchoscopy procedure is completed, the tube of the rigid bronchoscope is removed and an airway device, such as, for example, an Igel® supraglottic airway device, an endotracheal tube device or other type of airway device, is coupled via a breathing circuit to a ventilator machine to resume ventilation of the patient. During current flexible bronchoscopy procedures, such a mask or endotracheal tube is used and the flexible tube of the flexible bronchoscope is fed into the patient through an uncapped port of the airway device. When the flexible bronchoscopy procedure is complete, the flexible bronchoscope tube is removed from the patient and from the airway device and the uncapped port of the airway device is capped. Air delivered by the airway device to the patient continues to ventilate the patient until the patient is recovered.

As indicated above, the OPG 100 obviates the need for the additional airway device. In accordance with this representative embodiment, the tubular extension 101 is sized to mate with a known breathing tube of a breathing circuit of a known ventilator machine. Sizing the tubular extension 101 to mate with a known breathing tube allows the breathing tube that is coupled on one end to the ventilator machine to simply be coupled on its opposite end to the proximal end of the tubular extension 101 to allow ventilation of the patient to continue until the patient has recovered from anesthesia.

In accordance with a representative embodiment, the tubular extension 101 is tubular, i.e., cylindrical in shape, has an outer diameter and an inner diameter, a proximal end 101a that coincides with the proximal end 100a of the OPG 100, a distal end 101b, and a tubular section that extends from the proximal end 101a to the distal end 101b. The tubular section has a hollow inner bore defined by the inner diameter of the tubular extension 101. The proximal end 101a of the tubular extension 101 preferably is tapered and adapted to connect to the breathing tube of external equipment of the type discussed above used for providing air to patients' airways during post-procedure recovery from anesthesia. The outer diameter of the tubular extension 101 can be substantially equal to the inner diameter of the aforementioned breathing tube, or vice versa, to allow the extension 101 to easily mate with the end of the breathing tube in an airtight arrangement.

The inner diameter of the tubular extension 101 preferably is sufficiently large to accommodate the width or diameter of a rigid bronchoscope tube, at least in cases where the OPG 100 is intended to be used for rigid bronchoscopy and for cases where the OPG 100 is intended to be used for rigid and flexible bronchoscopy. Rigid bronchoscope tubes are larger in width or outer diameter than flexible bronchoscope tubes. For cases where the OPG 100 is intended to be used for flexible, but not rigid, bronchoscopy, the inner diameter of the extension 101 need only be large enough to accommodate a flexible bronchoscope tube. For purposes of describing the inventive principles and concepts of the present disclosure, it will be assumed that the OPG 100 is configured to be sufficiently versatile to be suitable for rigid and flexible bronchoscopy, and therefor that the extension 101 has an inner diameter that is sufficiently large to accommodate a rigid bronchoscope tube.

It should be noted that the inventive principles and concepts of the present disclosure are not limited to the tubular extension 101 having any particular dimensions or configuration. However, providing the tubular extension 101 with an outer diameter that is suitable for mating the extension 101 with a known breathing tube of a known ventilator machine breathing circuit reduces costs and complexity by eliminating the need to use an adapter to achieve an airtight interface between the breathing circuit tube and the proximal end 101a of the tubular extension 101. It should be noted, however, that it is within the scope of the inventive principles and concepts of the present disclosure to employ an adapter for such a purpose.

With reference to FIG. 2B, a teeth guard 102 of the OPG 100 comprises a relatively rigid hollow tube having a proximal end 102a that is coupled to or joined to the distal end 101b of the tubular extension 101. The teeth guard 102 has a teeth abutment portion 102d that is generally disc-shaped and has a generally oval circumference. The teeth abutment portion 102d extends outwardly from the outer surface of the relatively rigid hollow tube of the teeth guard 102. When the OPG 100 is in the installed position in the patient's mouth, the teeth abutment portion 102d is in abutment with front surfaces of the upper and lower front teeth of the patient to protect them. The teeth abutment portion 102d also acts as a stop to prevent the proximal end portion of the OPG 100 from moving passed the patient's upper and lower front teeth into the mouth of the patient. In this way, the teeth abutment portion 102d helps to properly position the proximal end portion of the OPG 100 for the bronchoscopy procedure.

A main tube 103 of the OPG 100 has a low-friction lumen to allow the rigid or flexible bronchoscope tube to easily move within it. The main tube 103 is slightly flexible to allow it to be slightly bent during installation of the OPG 100 in the patient's mouth and throat. A proximal end 103a of the main tube 103 is coupled to or joined to a distal end 102b of the relatively rigid hollow tube 102a of the teeth guard 102. The diameter of the lumen of the main tube 103, the inner diameter of the relatively rigid hollow tube of the teeth guard 102 and the inner diameter of the tubular extension 101 can be equal or substantially equal in size. The main tube 103, the relatively rigid hollow tube of the teeth guard 102 and the tubular extension 101 can be made of, for example, polyvinylchloride (PVC) or polyurethane (PU). It should be noted that the components of the OPG 100 are not limited to being made of any particular materials. Persons of skill in the art will understand how to choose suitable materials from which to make these components.

A teeth cushioning layer 104 comprising a relatively soft material such as, for example, flexible silicone, surrounds the outer surface of the relatively rigid hollow tube of the teeth guard 102 to provide a soft protective surface into which the upper and lower front teeth of the patient bite when the OPG 100 is in the installed position in the patient's mouth and throat. In the representative embodiment shown, the material comprising the teeth cushioning layer 104 also surrounds the outer surfaces of the main tube 103, at least along its length, although this is not required.

With reference to FIGS. 2B, 3B and 4, in accordance with this representative embodiment, the OPG 100 comprises first and second insufflatable bladders 105 and 106, respectively, that can be insufflated with air or another suitable gas via first and second insufflation tubes 107 and 108, respectively. It should be noted that while the OPG 100 is shown as having first and second insufflatable bladders, the OPG 100 may instead have single insufflatable bladder. The material comprising the bladder(s) can be any suitable airtight flexible material such as, for example, silicone. When insufflated, the first insufflatable bladder 105 acts as an oropharyngeal trajectory seal and the second insufflatable bladder 106 acts as a supraglottic airway seal. As indicated above, a single insufflatable bladder can perform both of these functions. In accordance with this representative embodiment, insufflation of the bladders 105 and 106 can be controlled independently with the separate insufflation tubes 107 and 108, respectively, which are used to insufflate the bladders 105 and 106, respectively. This allows the extent of insufflation of the bladders 105 and 106 to be precisely controlled to adjust for differences in anatomies of patients' mouths and throats to achieve airtight, or nearly airtight, seals.

An additional benefit of using the OPG 100 for anesthesia recovery is that when the OPG 100 is in the installed state in the patient's mouth, the partially or wholly insufflated bladder 106 seals off the upper aperture of the esophagus to prevent aspiration of gastric contents into the patient's airway while under anesthesia. By allowing the OPG 100 to remain in the installed state post-bronchoscopy during anesthesia recovery, aspiration of gastric contents is prevented while air is being provided to the patient's airway via the tubular extension 101.

With reference to FIG. 2C, in accordance with the representative embodiment, the distal end 100b of the OPG 100 has a flexible opening 111 that opens just wide enough to allow the tube of the bronchoscope (not shown) to pass through it to substantially seal the opening 111 to prevent air from passing through the opening.

As in the parent application, in accordance with a representative embodiment, the distal end 111 of the OPG 100 can be configured to be removably attached to enable differently configured distal ends to be used with the OPG 100 that are suited for rigid bronchoscopy or flexible bronchoscopy. Because the flexible tube of the flexible bronchoscope will typically not be sufficiently stiff to press through a very small, tight distal end 111, the distal end 111 can have a tubular or cylindrical shape and size similar to that of the tubular extension 101 through which the flexible bronchoscope tube can easily pass. In cases where the OPG 100 is being used for rigid bronchoscopy 100, the distal end 111 can be a slit or flexible opening that is smaller than the diameter of the bronchoscope tube such that it is stretched open when the bronchoscope tube passes through it and forms a tight fit around the bronchoscope tube that is airtight or nearly airtight.

When the OPG 100 is being used for rigid bronchoscopy and subsequent anesthesia recovery, a tube of the breathing circuit that is connected to the ventilator machine is connected on it opposite end to the proximal end 101a of the tubular extension 101 after the rigid bronchoscope tube has been removed from the OPG 100. This allows the OPG 100 to continue supplying air to the patient's airway. When the OPG 100 is being used for flexible bronchoscopy and anesthesia recovery, the tube of the breathing circuit that is connected to the ventilator machine is connected to the tubular extension 101 prior to performing the bronchoscopy procedure. After the breathing circuit is connected to the tubular extension, the tube of the flexible bronchoscope can be inserted via a port of the breathing circuit through the tubular extension 101 into the patient. Air is supplied to the patient through the tubular extension 101 during the bronchoscopy procedure and post procedure during anesthesia recovery. After the flexible bronchoscope procedure has been performed, the tube of the flexible bronchoscope can be retracted through the port of the breathing circuit and the port can be capped to prevent air leakage during recovery.

It should be noted that the inventive principles and concepts have been described with reference to representative embodiments, but that the inventive principles and concepts are not limited to the representative embodiments described herein. Although the inventive principles and concepts have been illustrated and described in detail in the drawings and in the foregoing description, such illustration and description are to be considered illustrative or exemplary and not restrictive; the invention is not limited to the disclosed embodiments. Other variations to the disclosed embodiments can be understood and effected by those skilled in the art, from a study of the drawings, the disclosure, and the appended claims.

What is claimed is:

1. An oropharyngeal glove (OPG) configured for use with a bronchoscope that is passed through the OPG into a patient's trachea during a bronchoscopy procedure, the OPG comprising:
a main tube having a proximal end, a distal end and a hollow inner lumen that extends from the proximal end to the distal end, the inner lumen being sized to allow a bronchoscope tube to be passed therethrough, the proximal end and the distal end having first and second openings, respectively, through which the bronchoscope tube can pass;
a flexible body secured about the main tube, the flexible body comprising at least a first insufflatable bladder configured to be insufflated to a state in which outer surfaces of said at least a first bladder form a seal with a patient's mouth and throat when the OPG is in an installed state in a patient's mouth and throat; and
a tubular extension having a proximal end, a distal end, and a hollow inner bore that extends from the proximal end of the tubular extension to the distal end of the tubular extension, the distal end of the tubular extension interfacing with the proximal end of the main tube, the hollow inner bore having an inner diameter that is sufficiently large to allow the tube of the bronchoscope to pass through the hollow inner bore and into the first opening of the main tube during a bronchoscopy procedure.

2. The OPG of claim 1, wherein the tubular extension is configured to couple with a ventilator machine via one or more tubes of a breathing circuit and to act as an airway device to deliver air produced by the ventilator machine to the patient after completion of the bronchoscopy procedure.

3. The OPG of claim 1, wherein said at least a first insufflatable bladder is configured such that when the OPG is in the installed state in the patient's mouth and throat and said at least a first insufflatable bladder is in a partially or wholly insufflated state, said seal acts to seal off an upper aperture of an esophagus of the patient to prevent aspiration of gastric contents from the esophagus into a trachea of the patient.

4. The OPG of claim 3, wherein said at least a first insufflatable bladder is configured such that when the OPG is in the installed state in the patient's mouth and throat and said at least a first insufflatable bladder is in a partially or wholly insufflated state, said seal acts to prevent or reduce air leakage from the trachea out of the mouth of the patient.

5. The OPG of claim 1, wherein the flexible body is configured to protect the patient's mouth and throat from being damaged by the bronchoscope tube during a bronchoscopy procedure

6. The OPG of claim 1, further comprising a teeth guard mechanically coupled to or joined to an exterior surface of the tubular extension, the teeth guard being configured to protect upper and lower teeth, respectively, of the patient, the teeth guard being in contact with front surfaces of upper and lower teeth of the patient when the OPG is in the installed state.

7. The OPG of claim 2, wherein the proximal end of the tubular extension has an outer surface with an outer diameter that is selected to interconnect with a connecting tube of said one or more tubes of the breathing circuit such that an airtight or substantially airtight seal exists between the outer surface of the proximal end of the tubular extension and an inner surface of the connecting tube.

8. The OPG of claim 2, wherein the proximal end of the tubular extension has an inner surface with an inner diameter that is selected to interconnect with a connecting tube of said one or more tubes of the breathing circuit such that an airtight or substantially airtight seal exists between the inner surface of the proximal end of the tubular extension and an outer surface of the connecting tube.

9. The OPG of claim 1, wherein said at least a first insufflatable bladder comprises at least first and second insufflatable bladders, and wherein the bladders can be insufflated to different levels independently of one another via first and second insufflation tubes, the first and second insufflation tubes having proximal ends that are exterior to the OPG for access by a user and distal ends that are disposed within the first and second insufflation bladders, respectively.

10. A method for performing a rigid or flexible bronchoscopy procedure and for performing anaesthesia recovery after the bronchoscopy procedure, the method comprising:
installing an oropharyngeal glove (OPG) in a patient's mouth and throat, the OPG comprising:
a main tube having a proximal end, a distal end and a hollow inner lumen that extends from the proximal end to the distal end, the inner lumen being sized to allow a bronchoscope tube to be passed therethrough, the proximal end and the distal end having first and second openings, respectively, through which the bronchoscope tube can pass;
a flexible body secured about the main tube, the flexible body comprising at least a first insufflatable bladder configured to be insufflated to a state in which outer surfaces of said at least a first insufflatable bladder form a seal with a patient's mouth and throat when the OPG is in an installed state in a patient's mouth and throat; and
a tubular extension having a proximal end, a distal end, and a hollow inner bore that extends from the proximal end of the tubular extension to the distal end of the tubular extension, the distal end of the tubular extension interfacing with the proximal end of the main tube, the hollow inner bore having an inner diameter that is sufficiently large to allow the tube of the bronchoscope to pass through the hollow inner bore and into the first opening of the main tube during a bronchoscopy procedure.
inserting a tube of a bronchoscope through the hollow inner bore, through the first and second openings and into a trachea of the patient; and
manipulating the tube of the bronchoscope to perform a bronchoscopy procedure.

11. The method of claim 10, further comprising:
after manipulating the tube of the bronchoscope to perform the bronchoscopy procedure:
removing the tube of the bronchoscope from the OPG; and
with a ventilator machine coupled via one or more tubes of a breathing circuit to the proximal end of the tubular extension, using the tubular extension as an airway device to deliver air produced by the ventilator machine to the patient to perform anaesthesia recovery.

12. The method of claim 10, further comprising:
after installing the OPG, insufflating said at least a first insufflatable bladder to adjust the seal.

13. The method of claim 10, wherein the tubular extension is configured to couple with a ventilator machine via one or more tubes of a breathing circuit and to act as an airway device to deliver air produced by the ventilator machine to the patient after completion of the bronchoscopy procedure.

14. The method of claim 10, wherein said at least a first insufflatable bladder is configured such that when the OPG is in the installed state in the patient's mouth and throat and said at least a first insufflatable bladder is in a partially or wholly insufflated state, said seal acts to seal off an upper aperture of an esophagus of the patient to prevent aspiration of gastric contents from the esophagus into a trachea of the patient.

15. The method of claim 14, wherein said at least a first insufflatable bladder is configured such that when the OPG is in the installed state in the patient's mouth and throat and said at least a first insufflatable bladder is in a partially or wholly insufflated state, said seal acts to prevent or reduce air leakage from the trachea out of the mouth of the patient.

16. The method of claim 10, wherein the flexible body is configured to protect the patient's mouth and throat from being damaged by the bronchoscope tube during a bronchoscopy procedure

17. The method of claim 10, further comprising a teeth guard mechanically coupled to or joined to an exterior surface of the tubular extension, the teeth guard being configured to protect upper and lower teeth, respectively, of the patient, the teeth guard being in contact with front surfaces of upper and lower teeth of the patient when the OPG is in the installed state.

18. The method of claim 11, wherein the proximal end of the tubular extension has an outer surface with a diameter that is selected to interconnect with a connecting tube of said one or more tubes of the breathing circuit such that an airtight or substantially airtight seal exists between the outer surface of the proximal end of the tubular extension and an inner surface of the connecting tube.

19. The method of claim 11, wherein the proximal end of the tubular extension has an inner surface with a diameter that is selected to interconnect with a connecting tube of said one or more tubes of the breathing circuit such that an airtight or substantially airtight seal exists between the inner surface of the proximal end of the tubular extension and an outer surface of the connecting tube.

20. The method of claim 10, wherein said at least a first insufflatable bladder comprises at least first and second insufflatable bladders, and wherein the bladders can be insufflated to different levels independently of one another via first and second insufflation tubes, the first and second insufflation tubes having proximal ends that are exterior to the OPG for access by a user and distal ends that are disposed within the first and second insufflation bladders, respectively.

21. The method of claim 19, wherein the bladders can be insufflated to different levels independently of one another via at least first and second insufflation tubes, the first and second insufflation tubes having proximal ends that are exterior to the OPG for access by a user and distal ends that are disposed within the first and second insufflation bladders, respectively.
